(19) United States
(12) Patent Application Publication
(10) Pub. No.: US 2023/0268031 A1
(43) Pub. Date: Aug. 24, 2023

(54) MONITORING AND MANAGEMENT OF CELL THERAPY-INDUCED TOXICITIES

(71) Applicant: Kite Pharma, Inc., Santa Monica, CA (US)

(72) Inventor: Qinghua Song, San Francisco, CA (US)

(21) Appl. No.: 17/816,076

(22) Filed: Jul. 29, 2022

Related U.S. Application Data
(60) Provisional application No. 63/279,615, filed on Nov. 15, 2021.
(60) Provisional application No. 63/227,677, filed on Jul. 30, 2021.

(51) Int. Cl. G16B 40/00; G16B 5/20

(52) U.S. Cl. CPC G16B 40/00; G16B 5/20

(57) ABSTRACT

The present disclosure relates generally to compositions and methods for identifying cell therapy patients as being likely or not likely to experience toxicity following the cell therapy. The methods are based on the discovery that pre-treatment covariates, such as serum IL-15 and MCP-1 levels in the patients or the viability of the cells being administered can be used predict the likelihood of the onset of such toxicities. Once the patient is identified as being likely or not likely to experience the toxicities, compositions and methods are also provided for monitoring and managing the toxicities.

### CROSS REFERENCE TO RELATED APPLICATIONS

This application claims priority to U.S. Provisional Pat. Application No. 63/227,677 filed on Jul. 30, 2021, and to U.S. Provisional Pat. Application No. 63/279,615 filed on Nov. 15, 2021; the entire contents of each of which is hereby incorporated by reference in its entirety.

### FIELD

The disclosure relates to methods for determining whether a patient is likely or not likely to experience toxicities following a cell therapy treatment.

### BACKGROUND

Chimeric antigen receptor T cells (also known as CAR T cells) are T cells that have been genetically engineered to produce an artificial T cell receptor for use in immunotherapy. CAR-T therapy has the potential to improve the management of lymphomas and possibly solid cancers. Two anti-CD19 CAR T-cell products, axicabtagene ciloleucel (axi-cel) and tisagenlecleucel, have been approved for the management of relapsed/refractory large B-cell lymphoma.

CAR-T therapies, however, are associated with two common toxicities, cytokine release syndrome (CRS) and immune effector cell-associated neurotoxicity syndrome (ICANS), which are typically observed acutely after the therapy. In addition, late toxicities include prolonged cytopenias and on-target off-tumor effects.

CRS is a systemic inflammatory response triggered by the release of cytokines by CAR-T cells following their activation upon tumor recognition. The CAR-T cells likely also activate bystander immune cells such as macrophages, which in turn release inflammatory cytokines and contribute to the pathophysiology of CRS. CRS typically occurs along with symptoms of fever, myalgias, rigors, fatigue, and loss of appetite. CRS can also lead to multiorgan dysfunction.

ICANS can occur during CRS or more commonly after CRS has subsided. It typically presents as a toxic encephalopathy with word-finding difficulty, aphasia, and confusion but can progress in more severe cases to depressed level of consciousness, coma, seizures, motor weakness, and cerebral edema. Cytokines, chemokines, and degree of CAR-T cell expansion have been associated with severity of neurotoxicity.

Monitoring for CRS and neurologic toxicities is required for patients receiving a CAR-T infusion. Given the potential severity of the toxicities, such monitoring is required to be done daily in a certified healthcare facility for 7 days. In addition, patients are instructed to remain within proximity of the certified healthcare facility for at least 4 weeks following infusion. Such monitoring results significant costs.

There is a strong need for methods to predict the onset of such toxicities, so that only those that require toxicity treatments need to remain on site, which can help reduce unnecessary hospital stays. Also, those predicted to likely experience the toxicities can receive appropriate treatment or prophylaxis for the toxicities.

### SUMMARY

The present disclosure provides compositions and methods for identifying cell therapy patients as being likely or not likely to experience toxicity following the cell therapy. The methods are based on the discovery that pre-treatment covariates, such as serum IL-15 and MCP-1 levels in the patients or the viability of the cells being administered can be used predict the likelihood of the onset of such toxicities. Once the patient is identified as being likely or not likely to experience the toxicities, compositions and methods are also provided for monitoring and managing the toxicities.

One embodiment provides a method for identifying a patient as being likely or not likely to experience toxicity following a cell therapy, comprising measuring the level of IL-15 (Interleukin-15) or MCP-1 (monocyte chemoattractant protein-1) in a blood sample of the patient, and identifying the patient as being likely to experience toxicity following the cell therapy when the IL-15 or MCP-1 level is higher than a corresponding reference level, or identifying the patient as being not likely to experience toxicity following the cell therapy when the IL-15 or MCP-1 level is lower than a corresponding reference level, wherein the cell therapy comprises administration of immune cells.

In some embodiments, the immune cells comprise T cells. In some embodiments, the T cells are engineered to express a chimeric antigen receptor (CAR). In some embodiments, the CAR has binding specificity to a CD19 (cluster of differentiation 19) protein. In some embodiments, the cell therapy comprises axicabtagene ciloleucel.

In some embodiments, the blood sample is a serum sample. In some embodiments, the blood sample is obtained from the patient prior to the cell therapy. In some embodiments, the blood sample is obtained following a preconditioning treatment of the patient. In some embodiments, the preconditioning treatment reduces lymphocytes in the patient. In some embodiments, the preconditioning comprises intravenous (iv) administration of cyclophosphamide and fludarabine given on the 5th, 4th, and/or 3rd day prior to the cell therapy.

In some embodiments, the toxicity is selected from the group consisting of cytokine release syndrome (CRS), neurologic events (NEs), and combinations thereof. In some embodiments, the toxicity is early onset toxicity. In some embodiments, the early onset toxicity occurs within four days following the cell therapy.

In some embodiments, the reference level for IL-15 or MCP-1 is determined from patients that experience the toxicity following the cell therapy and patients that do not experience the toxicity following the cell therapy.

In some embodiments, the method further comprises measuring viability of cells used in the cell therapy, wherein the patient is identified as being likely to experience toxicity following the cell therapy when the IL-15 or MCP-1 level is higher than the corresponding reference level and the cell viability is greater than a reference cell viability, or wherein the patient is identified as being not likely to experience toxicity following the cell therapy when the IL-15 or MCP-1 level is lower than the corresponding reference level and the cell viability is lower than the reference cell viability.

In some embodiments, the patient is identified as being likely to experience toxicity following the cell therapy when the IL-15 and MCP-1 level are higher than the corresponding reference levels and the cell viability is greater than the reference cell viability, or wherein the patient is identified as being not likely to experience toxicity following the cell therapy when the IL-15 and MCP-1 level are lower than the corresponding reference levels and the cell viability is lower than the reference cell viability.

In some embodiments, the method further comprises obtaining one or more levels of baseline hemoglobin, baseline tumor burden, baseline LDH, baseline creatinine, and baseline calcium of the patient.

In some embodiments, the method further comprises monitoring the patient for toxicity in a medical care facility, when the patient is identified as being likely to experience toxicity.

In some embodiments, the method further comprises preventing or treating the toxicity in the patient, when the patient is identified as being likely to experience toxicity. In some embodiments, the treatment or prevention comprises administration of an agent selected from the group consisting of anti-histamine, corticosteroid, antihypotensive agent, IL-6 inhibitor, GM-CSF inhibitor, and nonsteroidal anti-inflammatory drug. In some embodiments, the treatment or prevention comprises administration of an agent selected from the group consisting of tocilizumab, dexamethasone, levetiracetam, lenzilumab, methylprednisolone, anakinra, siltuximab, ruxolitinib, cyclophosphamide, IVIG (intravenous immunoglobulin) and ATG (antithymocyte globulin).

In some embodiments, the method further comprises releasing the patient from the medical care facility following the medical care facility within two days, when the patient is identified as being not likely to experience toxicity.

Also provided, in one embodiment, is a kit or package useful for identifying a patient as being likely to experience toxicity following a cell therapy, comprising polynucleotide primers or probes or antibodies for measuring the expression level of IL-15 and MCP-1 in a biological sample.

Also provided, in one embodiment, is a method for preventing or treating toxicity in a patient undergoing a cell therapy, comprising administering to the patient an agent that prevents or treats cytokine release syndrome (CRS) or neurologic events (NEs), wherein the patient has been identified as being likely to experience toxicity following the cell therapy based on level of IL-15 (Interleukin-15) or MCP-1 (monocyte chemoattractant protein-1) in a blood sample of the patient being higher than corresponding reference level.

In some embodiments, the agent is selected from the group consisting of anti-histamine, corticosteroid, antihypotensive agent, IL-6 inhibitor, GM-CSF inhibitor, and nonsteroidal anti-inflammatory drug. In some embodiments, the agent is selected from the group consisting of tocilizumab, dexamethasone, levetiracetam, lenzilumab, methylprednisolone, anakinra, siltuximab, ruxolitinib, cyclophosphamide, IVIG (intravenous immunoglobulin) and ATG (antithymocyte globulin).

Also provided, in one embodiment, is a computer program product for use in conjunction with a computer system, the computer program product comprising a computer readable storage medium and a computer program mechanism embedded therein, the computer mechanism comprising executable instructions for performing a method for identifying a patient as being likely to experience toxicity following a cell therapy, wherein the instructions comprise: (i) obtaining the level of IL-15 (Interleukin-15) or MCP-1 (monocyte chemoattractant protein-1) in a blood sample of the patient; and (ii) comparing the level to a corresponding reference level, wherein the patient is identified as being likely to experience toxicity following the cell therapy when the IL-15 or MCP-1 level is higher than the corresponding reference level, wherein the cell therapy comprises administration of immune cells.

### BRIEF DESCRIPTION OF THE DRAWINGS

FIG. 1 shows the patient conditions in Definition C.

FIG. 2 shows the ROC of the BPM with Cell viability + IL-15 + MCP-1 on outpatient A3. Here, the BPM is RFCRUS and the optimal cut-off is 0.538.

FIG. 3 shows a box plot of predictions on training data, BPM with Cell viability + IL-15 + MCP-1 on outpatient A3.

FIG. 4 shows a box plot of predictions on testing data, BPM with Cell viability + IL-15 + MCP-1 on outpatient A3.

FIG. 5 shows the decision tree on Cell viability + IL-15 + MCP-1 on training data with outpatient A3; subjects on leaves with “N” are classified as “inpatient”; subjects on leaves with “Y” are classified as “outpatient.”

FIG. 6 shows the decision tree on Cell viability + IL-15 + MCP-1 on testing data with outpatient A3; subjects on leaves with “N” are classified as “inpatient”; subjects on leaves with “Y” are classified as “outpatient.”

FIG. 7 shows a partial dependent plot (based on balanced RF) that shows higher Cell viability, IL-15 and MCP-1 are associated with higher likelihood of early onset toxicities.

FIG. 8 is a schematic illustrating the computing components that may be used to implement various features of the embodiments described in the present disclosure.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

All numerical designations, e.g., pH, temperature, time, concentration, and molecular weight, including ranges, are approximations which are varied ( + ) or ( - ) by increments of 0.1. It is to be understood, although not always explicitly stated that all numerical designations are preceded by the term “about”. The term “about” also includes the exact value “X” in addition to minor increments of “X” such as “X + 0.1” or “X - 0.1.” It also is to be understood, although not always explicitly stated, that the reagents described herein are merely exemplary and that equivalents of such are known in the art.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy may include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the conditioning methods disclosed herein would enhance the effectiveness of any transplanted T cell therapy. Examples of T cell therapies are described in U.S. Pat. Publication Nos. 2014/0154228 and 2002/0006409, U.S. Pat. No. 7,741,465, U.S. Pat. No. 6,319,494, U.S. Pat. No. 5,728,388, and International Publication No. WO 2008/081035. In some embodiments, the immunotherapy comprises CAR T cell treatment. In some embodiments, the CAR T cell treatment product is administered via infusion.

The T cells of the immunotherapy may come from any source known in the art. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Pat. Publication No. 2013/0287748, which is herein incorporated by reference in its entirety.

A “cytokine,” as used herein, refers to a non-antibody protein that is released by one cell in response to contact with a specific antigen, wherein the cytokine interacts with a second cell to mediate a response in the second cell. “Cytokine” as used herein is meant to refer to proteins released by one cell population that act on another cell as intercellular mediators. A cytokine may be endogenously expressed by a cell or administered to a subject. Cytokines may be released by immune cells, including macrophages, B cells, T cells, and mast cells to propagate an immune response. Cytokines may induce various responses in the recipient cell. Cytokines may include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1a), MIP-1β (MIP-1b), gamma-induced protein 10 (IP-10), and thymus and activation regulated chemokine (TARC or CCL17).

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell’s genome.

A “patient” as used herein includes any human who is afflicted with a cancer (e.g., a lymphoma or a leukemia). The terms “subject” and “patient” are used interchangeably herein.

The terms “reducing” and “decreasing” are used interchangeably herein and indicate any change that is less than the original. “Reducing” and “decreasing” are relative terms, requiring a comparison between pre- and post- measurements. “Reducing” and “decreasing” include complete depletions. Similarly, the term “increasing” indicates any change that is higher than the original value. “Increasing,” “higher,” and “lower” are relative terms, requiring a comparison between pre- and post- measurements and/or between reference standards. In some embodiments, the reference values are obtained from those of a general population, which could be a general population of patients. In some embodiments, the reference values come quartile analysis of a general patient population.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In another embodiment, “treatment” or “treating” includes a complete remission.

The disclosure further provides diagnostic, prognostic and therapeutic methods, which are based, at least in part, on determination of the expression level of a gene of interest identified herein.

For example, information obtained using the diagnostic assays described herein is useful for determining if a subject is likely suffering from a disease (e.g., cytokine release syndrome) or likely to develop the disease, or is suitable for a treatment. Based on the diagnostics/prognostic information, a doctor can recommend a therapeutic protocol.

As used throughout, the term “likely” refers to having a higher probability of occurring than not, or alternatively, of having a higher probability of occurring versus a predetermined control of average. By way of non-limiting example, a patient likely to experience toxicity following a cell therapy refers to that patient having a higher probability of experiencing toxicity than not. Alternatively, a patient likely to experience toxicity following a cell therapy refers to that patient having a higher statistical chance of experiencing toxicity as compared to the average occurrence of toxicity in a patient population treated with the cell therapy. One of ordinary skill in the art would recognize additional definitions in addition to the aforementioned.

It is to be understood that information obtained using the diagnostic assays described herein may be used alone or in combination with other information, such as, but not limited to, behavior assessment, genotypes or expression levels of other genes, clinical chemical parameters, histopathological parameters, or age, gender and weight of the subject.

### Prediction and Management of Early Onset Acute Toxicities

For cancer patients receiving current CAR-T treatments, daily monitoring for signs and symptoms of CRS and neurologic toxicities at a certified healthcare facility following the CAR-T infusion is required. Patients with Grade ≥3 cytokine release syndrome (CRS) and neurologic events (NEs) require intensive in-patient management.

With machine learning technology, the present disclosure describes compositions and methods for predicting early onset acute toxicities in patients that receive CAR-T treatments. Based on such prediction, the present disclosure also provides methods for preventing the toxicities in patients that are at risk of experiencing the toxicities, and treat the toxicities as needed.

As demonstrated in the examples, multivariate analysis and machine learning from data obtained from evaluable patients in patients involved in a clinical trial for a CAR-T therapy led to several comparable predictive models for early onset CRS or NEs, with best-performing models having ROC (receiver operating characteristic) AUC (area under the ROC curve) > 0.8 in training and > 0.7 in testing.

When used alone, each of these covariates independently correlated with the likelihood of developing the toxicities. Collectively, the predicating power is further increased. Example covariates include, without limitation, product cell viability (or simply cell viability), serum IL-15 level at Day 0 prior to infusion, and serum MCP-1 (CCL2) level at Day 0 prior to infusion. Additional example covariates include hemoglobin level, albumin level, red blood cell count, and ferritin level (Day 0 prior to infusion); blood concentrations (levels) of urate, calcium, phosphate, creatinine, chloride, LDH (lactate dehydrogenase), and IL-17 (at baseline); and red blood cell count, white blood cell count, neutrophil count, and basophil count (at baseline).

In accordance with one embodiment of the present disclosure, provided is a method for identifying a patient as being likely to experience toxicity following a cell therapy. In some embodiments, the method entails measuring the level of IL-15 (Interleukin-15) in a sample of the patient. It has been discovered herein that higher level of IL-15 correlates with higher incidence of toxicity following the cell therapy. Therefore, the method further entails identifying the patient as being likely to experience toxicity following the cell therapy when the IL-15 level is higher than a reference level (or cut-off level).

In accordance with one embodiment of the present disclosure, provided is a method for identifying a patient as being likely to experience toxicity following a cell therapy. In some embodiments, the method entails measuring the level of MCP-1 (monocyte chemoattractant protein-1) in a sample of the patient. It has been discovered herein that higher level of MCP-1 correlates with higher incidence of toxicity following the cell therapy. Therefore, the method further entails identifying the patient as being likely to experience toxicity following the cell therapy when the IL-15 level is higher than a reference level (or cut-off level).

In accordance with one embodiment of the present disclosure, provided is a method for identifying a patient as being likely to experience toxicity following a cell therapy. In some embodiments, the method entails measuring the viability of the cells. It has been discovered herein that higher viability of the cells being infused correlates with higher incidence of toxicity following the cell therapy. Therefore, the method further entails identifying the patient as being likely to experience toxicity following the cell therapy when the cell viability is higher than a reference level (or cut-off level).

In some embodiments, the measurement that is useful for predicting the onset of the toxicity is for any one or more of the following covariates: blood hemoglobin level, albumin level, red blood cell count, and ferritin level (Day 0 prior to infusion); blood concentrations (levels) of urate, calcium, phosphate, creatinine, chloride, LDH (lactate dehydrogenase), and IL-17 (at baseline); and red blood cell count, white blood cell count, neutrophil count, and basophil count (at baseline).

In some embodiments, the blood covariates (e.g., IL-15) are measured in a blood sample obtained from the patient. The blood sample, in some embodiments, is a serum sample.

The blood sample is obtained from the patient, in some embodiments, according to the designated time point. For instance, for baseline covariates, the blood sample is drawn before the cell therapy starts. For Day 0 covariates, the blood sample is drawn at Day 0, which is the day when the infusion is administered. In some embodiments, the blood sample is drawn before the infusion.

In some embodiments, the patient undergoes preconditioning treatments prior to the cell therapy; hence, Day 0 is after the preconditioning treatment. In some embodiments, the preconditioning is white blood cell- or lympho-depleting. An example lympho-depleting regimen consists of intravenous cyclophosphamide 500 mg/m² and fludarabine 30 mg/m², both given on the 5th, 4th, and 3rd day prior to initiation of the CAR-T infusion.

The reference levels (cut-off values) for IL-15 levels, MCP-1 levels, cell viabilities, of any of the above-mentioned covariates can be determined experimentally or from historical data, with methods known in the art. The reference level for each corresponding covariate can be determined before the measurement, or after the measurement. In some embodiments, the reference level is one that best separates (distinguishes) patients having different toxicity outcomes following the same cell therapy.

In some embodiments, the reference level is a particular number, such as 0.1 ng/mL. In some embodiments, however, the reference level is implicit in a plurality of reference standards. For instance, a measured level can be compared to a number of reference numbers, each is labeled with toxicity or no toxicity, using a nearest neighbor method. If the measured level is closer to reference levels associated with patients who experience toxicities, then the measured level predicts that the patient will likely experience toxicities as well. In this example, no particular reference level is derived from the reference numbers, but a comparison is effectively conducted.

In some embodiments, the reference level is implicit in a formula used to calculate a likelihood based on the measured level. For instance, linear or quadratic discriminant analysis formulas can be developed based on training data, and used to determine a probability number taking the measured level as input.

In some embodiments, the covariates can be used in combination. For instance, when the IL-15 level and MCP-1 level both are higher than corresponding reference levels, the patient is identified as being likely to experience toxicity following the cell therapy. In some embodiments, when the IL-15 level and cell viability both are higher than corresponding reference levels, the patient is identified as being likely to experience toxicity following the cell therapy. In some embodiments, when the MCP-1 level and cell viability both are higher than corresponding reference levels, the patient is identified as being likely to experience toxicity following the cell therapy. In some embodiments, when the IL-15 level, MCP-1 level and cell viability all are higher than corresponding reference levels, the patient is identified as being likely to experience toxicity following the cell therapy. In some embodiments, one or more of the additional covariates are also included.

In some embodiments, the reference level (plasma concentration) for IL-15 is 20 pg/mL, 21 pg/mL, 22 pg/mL, 23 pg/mL, 24 pg/mL, 25 pg/mL, 26 pg/mL, 27 pg/mL, 28 pg/mL, 29 pg/mL, 30 pg/mL, 31 pg/mL, 32 pg/mL, 33 pg/mL, 34 pg/mL, 35 pg/mL, 36 pg/mL, 37 pg/mL, 38 pg/mL, 39 pg/mL, 40 pg/mL, 41 pg/mL, 42 pg/mL, 43 pg/mL, 44 pg/mL, 45 pg/mL, 46 pg/mL, 47 pg/mL, 48 pg/mL, 49 pg/mL, or 50 pg/mL. In an example embodiment, the reference level for IL-15 is 28 pg/mL.

In some embodiments, the reference level (plasma concentration) for CCL2 is 600 pg/mL, 620 pg/mL, 640 pg/mL, 650 pg/mL, 660 pg/mL, 680 pg/mL, 700 pg/mL, 720 pg/mL, 740 pg/mL, 750 pg/mL, 760 pg/mL, 780 pg/mL, 800 pg/mL, 820 pg/mL, 840 pg/mL, 850 pg/mL, 860 pg/mL, 880 pg/mL, 900 pg/mL, 920 pg/mL, 940 pg/mL, 950 pg/mL, 960 pg/mL, 980 pg/mL, 1000 pg/mL, 1020 pg/mL, 1040 pg/mL, 1050 pg/mL, 1060 pg/mL, 1080 pg/mL, 1100 pg/mL, 1120 pg/mL, 1140 pg/mL, 1150 pg/mL, 1160 pg/mL, 1180 pg/mL, 1200 pg/mL, 1220 pg/mL, 1240 pg/mL, 1250 pg/mL, 1260 pg/mL, 1280 pg/mL, 1300 pg/mL, 1320 pg/mL, 1340 pg/mL, 1350 pg/mL, 1360 pg/mL, 1380 pg/mL, 1400 pg/mL, 1420 pg/mL, 1440 pg/mL, or 1450 pg/mL.

In some embodiments, the reference level for the product cell viability is 93%, 93.5%, 94%, 94.5%, 95%, 95.5%, 96%, 96.5% or 97%. In an example embodiment, the reference level for the product cell viability is 95%.

In some embodiments, the cell therapy is a therapy entailing administration of an immune cell. The immune cell, without limitation, can be a T cell, a natural killer (NK) cell, a monocyte, or a macrophage, without limitation.

In some embodiments, the immune cell is engineered to express a chimeric antigen receptor (CAR), resulting in production of CAR-T cells, CAR-NK cells, without limitation. In some embodiments, the CAR has binding specificity to a tumor antigen.

A “tumor antigen” is an antigenic substance produced in tumor cells, i.e., it triggers an immune response in the host. Tumor antigens are useful in identifying tumor cells and are potential candidates for use in cancer therapy. Normal proteins in the body are not antigenic. Certain proteins, however, are produced or overexpressed during tumorigenesis and thus appear “foreign” to the body. This may include normal proteins that are well sequestered from the immune system, proteins that are normally produced in extremely small quantities, proteins that are normally produced only in certain stages of development, or proteins whose structure is modified due to mutation.

An abundance of tumor antigens are known in the art and new tumor antigens can be readily identified by screening. Non-limiting examples of tumor antigens include EGFR, Her2, EpCAM, CD19, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP and Tenascin.

In some embodiments, the CAR has specificity to any of the tumor antigens discussed above, or to any one or more of CD19, CD20, CLL-1, TACI, MAGE, HPV-associated proteins, GPC-3, and BCMA. In some embodiments, the CAR has dual-specificity for two or more antigens (e.g. CD19 and CD20).

In some embodiments, the CAR has specificity to CD19 (cluster of differentiation 19). An example cell therapy that targets CD19 is axicabtagene ciloleucel. Axicabtagene ciloleucel, sold under the brand name Yescarta®, is a treatment for large B-cell lymphoma that has failed conventional treatment.

In some embodiments, the toxicity is selected from the group consisting of cytokine release syndrome (CRS), neurologic events (NEs), and combinations thereof. In some embodiments, the toxicity is early onset toxicity. In some embodiments, the early onset toxicity occurs within five days, four days, three days, or two days following the cell therapy.

Key manifestations of CRS include fever, hypotension, tachycardia, hypoxia, chills, and headache. Serious events that may be associated with CRS include cardiac arrhythmias (including atrial fibrillation and ventricular tachycardia), cardiac arrest, cardiac failure, renal insufficiency, capillary leak syndrome, hypotension, hypoxia, multi-organ failure and hemophagocytic lymphohistiocytosis/macrophage activation syndrome (HLH/MAS). CRS can be categorized into four different grades, Grades 1-4.

The most common neurologic toxicities include encephalopathy, headache, tremor, dizziness, delirium, aphasia, and insomnia. Serious events include leukoencephalopathy and seizures. Neurologic toxicities can be categorized into four different grades, Grades 1-4.

The patient can be identified as being likely to experience the toxicities, the type of toxicity, and the grade. Accordingly, monitoring, prevention and treatment can be provided to the patient.

At present, monitoring is required for all patient receiving CAR-T therapies in healthcare facilities, which leads to significant costs. With the instant technology, patients that are identified as not likely to experience the toxicities can be monitored at an outpatient capacity. Those identified as being likely to experience the toxicities can be monitored as inpatient.

Preventative and/or treatment measures can also be taken for those that are identified as being likely to experience the toxicities. Depending on the predicted toxicity, appropriate preventive/treatment measures can be taken. For instance, for predicted CRS, tocilizumab 8 mg/kg can be administered intravenously over 1 hour (not to exceed 800 mg). Alternatively, dexamethasone 10 mg can be administered intravenously once daily. Also, methylprednisolone can be used for more server CRS.

For predicted neurologic toxicities, tocilizumab, dexamethasone, levetiracetam, corticosteroids, and/or methylprednisolone can be used. Alternative preventive/treatment options include anakinra, siltuximab, ruxolitinib, cyclophosphamide, IVIG (intravenous immunoglobulin) and ATG (antithymocyte globulin).

It is also known that severe CRS can be prevented by anti-histamines or corticosteroids. Treatment for less severe CRS is supportive, addressing the symptoms like fever, muscle pain, or fatigue. Moderate CRS requires oxygen therapy and giving fluids and antihypotensive agents to raise blood pressure. For moderate to severe CRS, the use of immunosuppressive agents like corticosteroids may be useful.

IL-6 inhibitors (e.g., anti-IL-6 antibodies such as tocilizumab) are known to be useful for preventing/treating CRS. GM-CSF inhibitors (e.g., anti-GM-CSF antibodies, such as lenzilumab) may also be effective at preventing or managing cytokine release, by reducing activation of myeloid cells and decreasing the production of IL-1, IL-6, MCP-1, MIP-1, and IP-10.

Tocilizumab, dexamethasone, levetiracetam, lenzilumab, methylprednisolone, anakinra, siltuximab, ruxolitinib, cyclophosphamide, IVIG (intravenous immunoglobulin) and ATG (antithymocyte globulin).

An embodiment of the disclosure relates to a method for identifying a patient as being likely or not likely to experience toxicity following a cell therapy, comprising: measuring a level of at least one of IL-15 (Interleukin-15) and MCP-1 (monocyte chemoattractant protein-1) in a blood sample of the patient; and identifying the patient as being likely to experience toxicity following the cell therapy when the level of IL-15 or MCP-1 is higher than a corresponding reference level, or identifying the patient as being not likely to experience toxicity following the cell therapy when the IL-15 or MCP-1 level is lower than a corresponding reference level. In such an embodiment, the cell therapy comprises administration of immune cells.

An embodiment of the disclosure relates to the method above, further comprising preventing or treating the toxicity in the patient, when the patient is identified as being likely to experience toxicity.

An embodiment of the disclosure relates to the method above, where the treatment or prevention comprises administration of an agent selected from the group consisting of anti-histamine, corticosteroid, antihypotensive agent, IL-6 inhibitor, GM-CSF inhibitor, and nonsteroidal anti-inflammatory drug.

An embodiment of the disclosure relates to the method above, where the treatment or prevention comprises administration of an agent selected from the group consisting of tocilizumab, dexamethasone, levetiracetam, lenzilumab, methylprednisolone, anakinra, siltuximab, ruxolitinib, cyclophosphamide, IVIG (intravenous immunoglobulin) and ATG (antithymocyte globulin).

An embodiment of the disclosure relates to the method above, where the immune cells comprise T cells engineered to express a chimeric antigen receptor (CAR).

An embodiment of the disclosure relates to the method above, where the CAR has binding specificity to a CD19 (cluster of differentiation 19) protein.

An embodiment of the disclosure relates to the method above, where the blood sample is a serum sample obtained from the patient prior to the cell therapy.

An embodiment of the disclosure relates to the method above, where the blood sample is obtained following a preconditioning treatment of the patient.

An embodiment of the disclosure relates to the method above, where the preconditioning treatment reduces lymphocytes in the patient.

An embodiment of the disclosure relates to the method above, where the toxicity is selected from the group consisting of cytokine release syndrome (CRS), neurologic events (NEs), and combinations thereof.

An embodiment of the disclosure relates to the method above, where the toxicity is early onset toxicity.

An embodiment of the disclosure relates to the method above, where the early onset toxicity occurs within four days following the cell therapy.

An embodiment of the disclosure relates to the method above, where the reference level for IL-15 or MCP-1 is determined from patients that experience the toxicity following the cell therapy and patients that do not experience the toxicity following the cell therapy.

An embodiment of the disclosure relates to the method above, further comprising measuring viability of cells used in the cell therapy, wherein the patient is identified as being likely to experience toxicity following the cell therapy when the IL-15 or MCP-1 level is higher than the corresponding reference level and the cell viability is greater than a reference cell viability, or wherein the patient is identified as being not likely to experience toxicity following the cell therapy when the IL-15 or MCP-1 level is lower than the corresponding reference level and the cell viability is lower than the reference cell viability.

An embodiment of the disclosure relates to the method above, further comprising obtaining one or more levels of baseline hemoglobin, baseline tumor burden, baseline LDH, baseline creatinine, and baseline calcium of the patient.

An embodiment of the disclosure relates a method for preventing or treating toxicity in a patient undergoing a cell therapy, comprising: identifying the patient as being likely or not likely to experience toxicity following a cell therapy, comprising: measuring a level of at least one of IL-15 (Interleukin-15) and MCP-1 (monocyte chemoattractant protein-1) in a blood sample of the patient; and identifying the patient as being likely to experience toxicity following the cell therapy when the level of IL-15 or MCP-1 is higher than a corresponding reference level, or identifying the patient as being not likely to experience toxicity following the cell therapy when the IL-15 or MCP-1 level is lower than a corresponding reference level. In such an embodiment, administering to the patient an agent that prevents or treats cytokine release syndrome (CRS) or neurologic events (NEs) if the patient has been identified as being likely to experience toxicity following the cell therapy.

An embodiment of the disclosure relates to the method above, where the agent is selected from the group consisting of anti-histamine, corticosteroid, antihypotensive agent, IL-6 inhibitor, GM-CSF inhibitor, and nonsteroidal anti-inflammatory drug.

An embodiment of the disclosure relates to the method above, where the agent is selected from the group consisting of tocilizumab, dexamethasone, levetiracetam, lenzilumab, methylprednisolone, anakinra, siltuximab, ruxolitinib, cyclophosphamide, IVIG (intravenous immunoglobulin) and ATG (antithymocyte globulin).

An embodiment of the disclosure relates to the method above, further comprising measuring viability of cells used in the cell therapy, wherein the patient is identified as being likely to experience toxicity following the cell therapy when the IL-15 or MCP-1 level is higher than the corresponding reference level and the cell viability is greater than a reference cell viability, or wherein the patient is identified as being not likely to experience toxicity following the cell therapy when the IL-15 or MCP-1 level is lower than the corresponding reference level and the cell viability is lower than the reference cell viability.

An embodiment of the disclosure relates to the method above, further comprising obtaining one or more levels of baseline hemoglobin, baseline tumor burden, baseline LDH, baseline creatinine, and baseline calcium of the patient.

### Kits and Packages, Software Programs

The methods described herein may be performed, for example, by utilizing prepackaged diagnostic kits, such as those described below, comprising at least one probe or primer nucleic acid described herein, which may be conveniently used, e.g., to determine whether a subject has or is at risk of experiencing toxicity following a cell therapy.

Accordingly, an embodiment of the disclosure relates to a kit or package useful for identifying a patient as being likely to experience toxicity following a cell therapy, comprising polynucleotide primers or probes or antibodies for measuring the expression level of IL-15 and MCP-1 in a biological sample.

Diagnostic procedures can be performed with mRNA isolated from cells or in situ directly upon tissue sections (fixed and/or frozen) of primary tissue such as biopsies obtained from biopsies or resections, such that no nucleic acid purification is necessary. Nucleic acid reagents can be used as probes and/or primers for such in situ procedures.

In one embodiment, provided is a kit or package useful for identifying a patient as being likely or not likely to experience toxicity following a cell therapy, comprising polynucleotide primers or probes or antibodies for measuring the expression level of IL-15 and MCP-1 in a biological sample. In some embodiments, the kit or package further includes agents for measuring the viability of the cells.

In one embodiment, a kit further includes instructions for use. In one aspect, a kit includes a manual comprising reference gene expression levels.

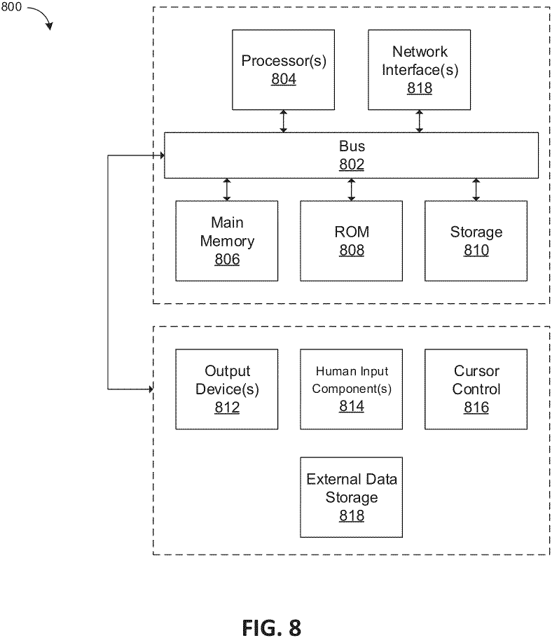

FIG. 8 is a block diagram that illustrates a computer system 800 upon which any embodiments of the present and related technologies may be implemented. The computer system 800 includes a bus 802 or other communication mechanism for communicating information, one or more hardware processors 804 coupled with bus 802 for processing information. Hardware processor(s) 804 may be, for example, one or more general purpose microprocessors.

The computer system 800 also includes a main memory 806, such as a random access memory (RAM), cache and/or other dynamic storage devices, coupled to bus 802 for storing information and instructions to be executed by processor 804. Main memory 806 also may be used for storing temporary variables or other intermediate information during execution of instructions to be executed by processor 804. Such instructions, when stored in storage media accessible to processor 804, render computer system 800 into a special-purpose machine that is customized to perform the operations specified in the instructions.

The computer system 800 further includes a read only memory (ROM) 808 or other static storage device coupled to bus 802 for storing static information and instructions for processor 804. A storage device 810, such as a magnetic disk, optical disk, or USB thumb drive (Flash drive), etc., is provided and coupled to bus 802 for storing information and instructions.

The computer system 800 may be coupled via bus 802 to a display 812, such as a LED or LCD display (or touch screen), for displaying information to a computer user. An input device 814, including alphanumeric and other keys, is coupled to bus 802 for communicating information and command selections to processor 804. Another type of user input device is cursor control 816, such as a mouse, a trackball, or cursor direction keys for communicating direction information and command selections to processor 804 and for controlling cursor movement on display 812. In some embodiments, the same direction information and command selections as cursor control may be implemented via receiving touches on a touch screen without a cursor. Additional data may be retrieved from the external data storage 818.

The computer system 800 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and maybe originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein are preferably implemented as software modules, but may be represented in hardware or firmware. Generally, the modules described herein refer to logical modules that may be combined with other modules or divided into sub-modules despite their physical organization or storage. In some embodiments, coding for desired analyses is conducted in R Core Team (2019); a language and environment for statistical computing (R Foundation for Statistical Computing, Vienna, Austria).

The computer system 800 may implement the techniques described herein using customized hard-wired logic, one or more ASICs or FPGAs, firmware and/or program logic which in combination with the computer system causes or programs computer system 800 to be a special-purpose machine. According to one embodiment, the techniques herein are performed by computer system 800 in response to processor(s) 804 executing one or more sequences of one or more instructions contained in main memory 806. Such instructions may be read into main memory 806 from another storage medium, such as storage device 810. Execution of the sequences of instructions contained in main memory 806 causes processor(s) 804 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 810. Volatile media includes dynamic memory, such as main memory 806. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 802. Transmission media can also take the form of acoustic or light waves, such as those generated during radio-wave and infra-red data communications.

Various forms of media may be involved in carrying one or more sequences of one or more instructions to processor 804 for execution. For example, the instructions may initially be carried on a magnetic disk or solid-state drive of a remote computer. The remote computer can load the instructions into its dynamic memory and send the instructions over a telephone line using a component control. A component control local to computer system 800 can receive the data on the telephone line and use an infra-red transmitter to convert the data to an infra-red signal. An infra-red detector can receive the data carried in the infra-red signal and appropriate circuitry can place the data on bus 802. Bus 802 carries the data to main memory 806, from which processor 804 retrieves and executes the instructions. The instructions received by main memory 806 may retrieve and execute the instructions. The instructions received by main memory 806 may optionally be stored on storage device 810 either before or after execution by processor 804.

The computer system 800 also includes a communication interface 818 coupled to bus 802. Communication interface 818 provides a two-way data communication coupling to one or more network links that are connected to one or more local networks. For example, communication interface 818 may be an integrated services digital network (ISDN) card, cable component control, satellite component control, or a component control to provide a data communication connection to a corresponding type of telephone line. As another example, communication interface 818 may be a local area network (LAN) card to provide a data communication connection to a compatible LAN (or WAN component to communicated with a WAN). Wireless links may also be implemented. In any such implementation, communication interface 818 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world-wide packet data communication network now commonly referred to as the “Internet”. Local network and Internet both use electrical, electromagnetic or optical signals that carry digital data streams. The signals through the various networks and the signals on network link and through communication interface 818, which carry the digital data to and from computer system 800, are example forms of transmission media.

The computer system 800 can send messages and receive data, including program code, through the network(s), network link and communication interface 818. In the Internet example, a server might transmit a requested code for an application program through the Internet, the ISP, the local network and the communication interface 818.

The received code may be executed by processor 804 as it is received, and/or stored in storage device 810, or other non-volatile storage for later execution. Each of the processes, methods, and algorithms described in the preceding sections may be embodied in, and fully or partially automated by, code modules executed by one or more computer systems or computer processors comprising computer hardware. The processes and algorithms may be implemented partially or wholly in application-specific circuitry.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the embodiments should, therefore, be construed in accordance with the appended claims and any equivalents thereof.

The various operations of example methods described herein may be performed, at least partially, by one or more processors that are temporarily configured (e.g., by software) or permanently configured to perform the relevant operations. Similarly, the methods described herein may be at least partially processor-implemented, with a particular processor or processors being an example of hardware. For example, at least some of the operations of a method may be performed by one or more processors. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)).

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Prediction of Early Onset Cytokine Release Syndrome and Neurologic Events After Axicabtagene Ciloleucel in Large B Cell Lymphoma Based on Machine Learning Algorithms

In clinical trial ZUMA-1, the pivotal study of axicabtagene ciloleucel (axi-cel) in patients with refractory large B-cell lymphoma (LBCL), Grade ≥3 cytokine release syndrome (CRS) and neurologic events (NEs) occurred in 13% and 28% of patients, respectively, and required intensive in-patient management. With increased safety experience, the management of CRS and NEs has been under evaluation in several exploratory safety management cohorts of ZUMA-1. Cohort 4 evaluated levetiracetam prophylaxis and earlier corticosteroid and/or tocilizumab use on the incidence and severity of CRS and NEs. The impact of adding prophylactic corticosteroids to the Cohort 4 toxicity management regimen was assessed in Cohort 6. Notably, some treated patients have early versus late onset of CRS or NEs, warranting distinct management. To facilitate toxicity management, this example developed predictive algorithms for early onset acute toxicities (within 3 –4 days after axi-cel) based on machine learning from ZUMA-1 data.

Methods: This post hoc analysis included patients from ZUMA-1 Phase 1 and Phase 2 Cohorts 1, 2, 4, and 6. Covariates (>1500; 227 measured before axi-cel infusion) included baseline product, patient and tumor characteristics, and proinflammatory soluble blood biomarker levels. Data from patients in Cohorts 1, 2, and 4 were randomly divided into training (70%) and testing (30%) sets. Univariate and multivariate analyses and clinical feasibility considerations were applied to select a covariate subset for further analysis. Machine learning (e.g., logistic regression, random forest, XGBoost, and AdaBoost classifier) was applied to 3 categories of covariates (1, clinical; 2, mechanistic [e.g., product attributes, inflammatory blood biomarkers]; 3, hybrid of 1 and 2) to build best-performing models (predictive performance evaluated by area under the curve [AUC] on test data). Optimal cutoffs for predictive scores were selected by receiver operating characteristic (ROC) or classification tree analysis. Data from patients in Cohort 6 were included to validate the best-performing model generated using training data.

Results: Multivariate analysis and machine learning from data obtained from 149 evaluable patients in ZUMA-1 Cohorts 1, 2, and 4 led to several comparable predictive models for early onset CRS or NEs (best-performing models with ROC AUC >0.8 in training and >0.7 in testing). The covariates in best-performing models included product cell viability, centrally measured Day 0 (before axi-cel treatment) IL-15 and CCL2 (MCP-1) serum levels and locally measured blood cell counts, blood chemistry analytes, tumor burden, and serum lactate dehydrogenase level. Best-performing models with <5 covariates contained only mechanistic covariates or a hybrid mix of covariates. A 3-covariate mechanistic model (product cell viability and Day 0 IL-15 and CCL2 (MCP-1) serum levels, all positively associated with early onset toxicities) performed comparably (ROC AUC >0.7 in testing) to larger best-performing models. Classification trees with splitting based on Day 0 IL-15 and product cell viability showed a potential to categorize patients by early versus late onset of toxicities (specificity >0.85).

Machine learning applied to covariates measured before axi-cel infusion yielded predictive models for early onset CRS or NEs that can be used for toxicity prediction, monitoring, and management. High performing hybrid or mechanistic models corroborated the importance of T-cell viability (product cell fitness) and conditioning-related elevation of factors (IL-15 and CCL2) that influence toxicities.

### Example 2: Prediction of Early Onset Cytokine Release Syndrome and Neurologic Events

This Example describes the data which were used to build the algorithms in Example 1, and the procedures of the developing the predictive algorithms, including: feature screening and selection, multivariate modeling, model evaluation, and classification on test population by predictive algorithms.

### Data

All analyses were performed in ZUMA1 patients’ safety analysis set (i.e. received any amount of axicabtagene ciloleucel) with cutoff date 06 Nov. 2019.

The populations included (a) Phase 1, and cohort 1 and cohort 2 in Phase 2, as of the 36 month cutoff (Phase 1 had 7 subjects with DLBCL, PMBCL, or TFL; Phase 2 cohort 1 had 77 subjects with refractory DLBCL; Phase 2 cohort 2 had 24 subjects with refractory PMBCL and TFL); (b) Phase 2 cohort 3 (38 subjects with relapsed or refractory transplant ineligible DLBCL, PMBCL, or TFL); (c) Phase 2 cohort 4 (41 subjects with relapsed or refractory DLBCL, PMBCL, TFL or HGBCL after 2 or more lines of systemic therapy).

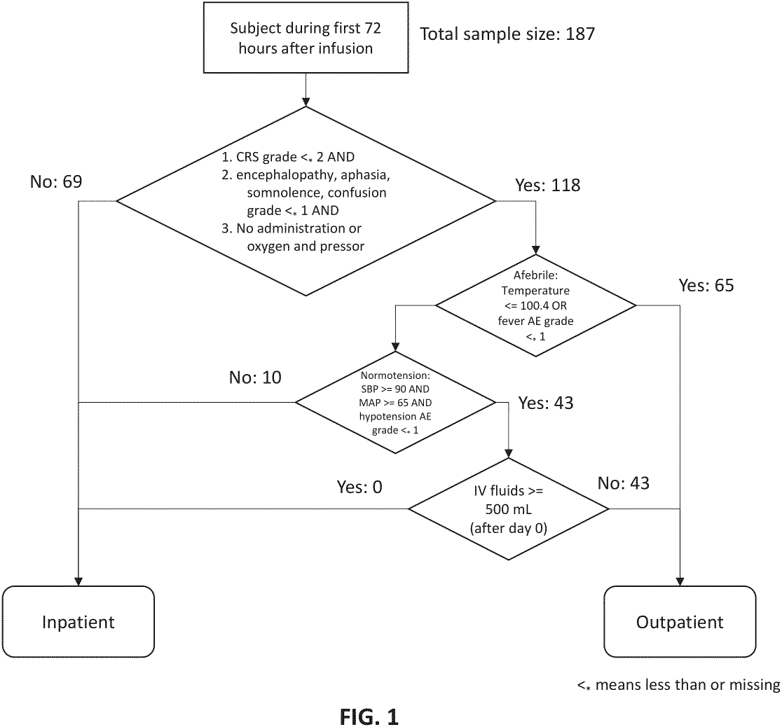

The following time windows were considered: 1, Day 0, 1, 2; 2, Day 0, 1, 2, 3; and Day 0, 1, 2, 3, 4. For each of the above time windows, three outpatient definitions were defined (see FIG. 1 and Table 1):
Definition A: Patients satisfying both (a) worst grade 1 or none of CRS (i.e., CRS worst grade <= 1), and (b) none of neurologic events (NE) during given time window;
Definition B: Patients with none of CRS or NE onset during given time window;
Definition C (proposed by Medical Affair and Clinical Research).

Patients who did not meet the above “Outpatient” criteria were assigned as “Inpatient” for each definition, respectively.

TABLE 1

Outpatient Definitions

| Outpatient | Definition | All Cohorts (# outpatient / # total) | Phase 1 Phase 2 C1&C2 | Phase 2 C3 | Phase 2 C4 |
|---|---|---|---|---|---|
| Definition A2 (Day 0 to 2) | Patients with (a) worst grade 1 or None of CRS, and (b) None of Neurologic events (NE) | 112 / 187 (60%) | 65 / 108 (60%) | 24 / 38 (63%) | 23 / 41 (56%) |
| Definition A3 (Day 0 to 3) | | 92 / 187 (49%) | 54 / 108 (50%) | 20 / 38 (53%) | 18 / 41 (44%) |
| Definition A4 (Day 0 to 4) | | 75 / 187 (40%) | 43 / 108 (40%) | 18 / 38 (47%) | 14 / 41 (34%) |
| Definition B2 (Day 0 to 2) | Patients with None of CRS or NE onset | 50 / 187 (27%) | 27 / 108 (25%) | 9 / 38 (24%) | 14 / 41 (34%) |
| Definition B3 (Day 0 to 3) | | 39 / 187 (21%) | 20 / 108 (19%) | 8 / 38 (21%) | 11 / 41 (27%) |
| Definition B4 (Day 0 to 4) | | 27 / 187 (14%) | 12 / 108 (11%) | 4 / 38 (11%) | 11 / 41 (27%) |
| Definition C2 (Day 0 to 2) | Patients identified by AE, vital sign, intervention (Details on right) | 108 / 187 (58%) | 61 / 108 (56%) | 24 / 38 (63%) | 23 / 41 (56%) |
| Definition C3 (Day 0 to 3) | | 83 / 187 (44%) | 48 / 108 (44%) | 19 / 38 (50%) | 16 / 41 (39%) |
| Definition C4 (Day 0 to 4) | | 66 / 187 (35%) | 36 / 108 (33%) | 16 / 38 (42%) | 14 / 41 (34%) |

Note: In definition C, a time window is a condition of the definition of “outpatient” or “inpatient”. For example, if Day 0 to 2 is given, then all criteria will be checked within Day 0, 1, 2 after infusion.

### Covariates and Feature Selection

Covariates (or more than 1500; 227 measured pre-axi-cel infusion) included baseline product, patient and tumor characteristics, and proinflammatory soluble blood biomarker levels. The major categories of the covariates or analytes included:
Baseline characteristics, such as ECOG performance, disease type, disease stage, International prognostic index (IPI) category, tumor burden, etc;
Lab analytes in both chemistry and hematology;
Serum cytokines and inflammatory markers;
Product characteristics, including product cell viability, number and percentage of CD4 and CD8, as well as CD4/CD8 ratio, phenotypes/re-gated phenotypes on CD4 and CD8, IFN-gamma in co-culture, etc; and
Cell growth information, including cell doubling time (in days) and expansion rate.

The data were randomly split into a training set (e.g., 70% of samples) to fit the model and to use a test set (e.g., the remaining 30% of samples) to provide an unbiased evaluation of model performance.

### Univariate Screening

Univariate analysis of each covariate was conducted one at a time, in which a covariate’s association with outpatient/inpatient status is evaluated, and those variables that pass screening criteria are selected and used in the multivariate modeling.

### Feature Selection by Analytical Approach

After K-Nearest Neighbors (KNN) imputation was performed for missing data, the following statistical- and model-based approaches were applied to the features which pass the univariate screening. Features were ranked and top-ranked features are selected by each of these approaches. Features that were selected by three, four, or all five of the methods described below, may be considered as “analytically important” features.

Weight of Evidence & Information Value: Weight of evidence (WOE) + information value (IV) is a simple method used to estimate the predictive power of a feature for an outcome of interest. WOE splits the data for each feature into several bins, e.g., j=10 bins, and calculates the predictive power (i.e., the “evidence”) of the feature for the outcome within each bin. For each feature, IV then combines the WOEs of all bins into a single score which is calculated as: IV = ∑ⱼ (proportion of non-eventsⱼ - proportion of eventsⱼ) * WOEⱼ. Features with higher IV values are selected as candidates for machine learning model (for example, IV values >= 0.3 or IV values >= 0.5 are considered “moderately good” or “good”, respectively.)

SelectkBest with Analysis of Variance: SelectkBest is a univariate feature selection method used to identify features that best explain the outcome. Specifically, for each feature analysis of variance (ANOVA) was performed and the corresponding F-statistic representing the ratio of explained to unexplained variation between the feature and the outcome was computed. The SelectKBest function then selected features with the k highest scores, e.g., lowest p-values, as the “best” features.

Extra Trees Classifier: The extra trees classifier (also known as extremely randomized trees) is a type of ensemble learning technique that aggregates the results of many decorrelated decision trees into a “forest” to output a classification result. A Gini Importance can be used to select features with highest importance (e.g., 30 features) in predicting the outcome.

Recursive Feature Elimination (RFE): Recursive feature elimination (RFE) was applied to a fitted model that has importance weights assigned to features (e.g., model coefficients, importance attributes) and eliminates the worst performing features for the model until the desired number of features is achieved. The top-ranked features, e.g., 30 features, may be selected for model building.

RFE-based Logistic Regression: RFE was applied to a logistic regression model, with variable importance defined by model coefficients.

RFE-based Random Forest: RFE was applied to a model estimated using random forest, with splits determined using a specific criterion (e.g., Gini index is used as a default) and variable importance evaluated using feature importance scores.

### Feature Selection by SME (Subject Matter of Experts)

SME (Subject Matter of Experts) review the list of analytically important features from the univariate and multivariate approaches, consider the clinical feasibility and provide 3 categories of covariates for further analyses:
Clinical Covariates. For example, tumor related (LDH, burden), disease stage, blood cell counts (WBC, RBC), analytes related to cells (Hgb), analytes related to metabolic status;
Mechanic Covariates. For example, product cell viability, day 0 IL-15, day 0 MCP-1, cytokines, chemokines, and other product attributes; and
Hybrid (Clinical + Mechanic) Covariates.

Lists of covariates were generated as imported candidates for classification model building.

### Multivariate Modeling With Machine Learning Algorithms

Five Machine Learning algorithms were applied on the covariates in each of these lists (Clinical Covariates, Mechanic Covariates, and Hybrid). All classification algorithms rely on a set of hyperparameters, which are “tuned” to find the combination that yields optimal performance. The model with the best predictive performance among the five machine learning algorithms was considered as the Best Performance Model (BPM). The simple descriptions of these Machine Learning algorithms are as follow:
Logistic Regression: Logistic regression is a parametric method that models the log odds of the probability of a binary event occurring as a linear combination of features. In our approach, we use a random under-sampled dataset fed into the logistic regression algorithm, which we call LOGREGRUS (Logistic Regression with Random Under Sampling).
Random Forest: Random Forest is an ensemble learning method designed to reduce the variance that can result from a single model (i.e., a decision tree). Random forest classification utilizes bootstrap aggregating (bagging), a technique that first bootstraps the training data, makes predictions, and then aggregates the results from the individual models to make more accurate predictions overall. This example used a random under-sampled dataset fed into the random forest algorithm, referred to as RFCRUS (Random Forest Classifier with Random Under Sampling).
Extreme Gradient Boosting (XGBoost): Boosting is an ensemble machine learning technique in which many weak learners (e.g., decision trees) are combined iteratively to form a final strong learner. Models are added sequentially until no further improvements can be made. Gradient boosting refers to the implementation of boosting using an arbitrary differentiable loss function and gradient descent optimization algorithm. Extreme gradient boosting refers to a quick and efficient implementation of the gradient boosting algorithm. This example used a random under-sampled dataset fed into the XGBoost, referred to as XGBCRUS (XGBoost Classifier with Random Under Sampling).
Balanced Random Forest Classifier (BRFC): The balanced random forest classifier (BRFC) differs from the random forest classifier in that it uses balanced bootstrap samples of training data. It differs from a random under-sampled dataset fed into the random forest algorithm because it does not preprocess the training data prior to learning a random forest classifier.
Random Under-sampling Boost Classifier (RUSBoost): Adaptive boosting (AdaBoost) is an ensemble boosting machine learning method that seeks to combine multiple weak classifiers (i.e., decision stumps) into a single strong classifier. It adaptively reweights the training samples based on classifications from previous learners, with larger weights given to misclassified samples. The final prediction is a weighted average of all the weak learners, with more weight placed on strong learners. Random Under-Sampling Boost (RUSBoost) adapts AdaBoost to the case with imbalanced data, by random under-sampling at each iteration of the boosting algorithm.

### Model Evaluation

Receiver Operating Characteristic (ROC) and AUC: The receiver operating characteristic (ROC) curve is a method for evaluating and comparing the performance of classification models. The false positive and true positive rates for a classifier are evaluated across a grid of possible (predicted probability) cut points defining whether an observation is classified as an event or a nonevent and these values are plotted. The area under the ROC curve (AUC) can also be calculated.

Tables 2-6 show the selected covariates and AUCs from the BPM, where BPM is selected as the one with highest AUC from testing data, among five machine learning algorithms.

TABLE 2

Selected covariates from Hybrid Models

| Product attributes | Patient /Tumor characteristics | Blood chemistry | | Blood cells | | Inflammatory markers | |
|---|---|---|---|---|---|---|---|
| | | Baseline | Day 0 | Baseline | Day 0 | Baseline | Day 0 |
| Cell viability↓ Total cells↨ | Bulky disease↑ | Urate↑ Calcium↓ Phosphate↑ Creatinine↓ Chloride ↑ LDH↕ | Albumin↑ | RBC↑ WBC↓ Neutrophils↓ Basophils↑ | RBC↑ Hgb↑ | IL-17↨↕ | IL-15↓ MCP-1↓ Ferritin↨ |

Covariates that are positively and negatively associated with all 9 outpatient definitions are indicated with ↑ and ↓, respectively. Covariates that had different association directions across the 9 outpatient definitions are shown with ↕. Models that make predictions that are 100% correct have AUC values equal to 1.

TABLE 3

AUCs of the BPMs based on selected covariates for Hybrid Models

| Outpatient Definition | Train AUC | Test AUC |
|---|---|---|
| A2 (RFCRUS) | 0.93 | 0.716 |
| B2 (XGBCRUS) | 0.948 | 0.779 |
| C2 (LOGREGRUS) | 0.757 | 0.715 |
| A3 (RUSBoost) | 0.945 | 0.712 |
| B3 (BRFC) | 0.988 | 0.684 |
| C3 (RFCRUS) | 0.879 | 0.647 |
| A4 (LOGREGRUS) | 0.831 | 0.777 |
| B4 (RFCRUS) | 1 | 0.748 |
| C4 (RUSBoost) | 0.897 | 0.668 |

TABLE 4

Selected covariates from Minimalistic Hybrid Models

| Product attributes | Patient /Tumor characteristics | Blood chemistry | | Blood cells | | Inflammatory markers | |
|---|---|---|---|---|---|---|---|
| | | Baseline | Day 0 | Baseline | Day 0 | Baseline | Day 0 |
| Cell viability↓ | | Urate↑ Calcium↓ | | RBC↑ | | | IL-15↓ MCP-1↓ |

Covariates that are positively and negatively associated with all 9 outpatient definitions are indicated with ↑ and ↓, respectively. Models that make predictions that are 100% correct have AUC values equal to 1.

TABLE 5

AUCs of the BPMs based on selected covariates for Minimalistic Hybrid Models

| Outpatient Definition | Train AUC | Test AUC |
|---|---|---|
| A2 (RFCRUS) | 0.867 | 0.737 |
| B2 (RFCRUS) | 0.914 | 0.669 |
| C2 (RUSBoost) | 0.839 | 0.633 |
| A3 (RUSBoost) | 0.854 | 0.736 |
| B3 (XGBCRUS) | 0.873 | 0.688 |
| C3 (XGBCRUS) | 0.785 | 0.77 |
| A4 (RFCRUS) | 0.899 | 0.741 |
| B4 (RFCRUS) | 1 | 0.878 |
| C4 (RFCRUS) | 0.903 | 0.638 |

TABLE 6

Selected Covariates and AUCs from Minimalistic Mechanistic Model and addition of selected clinical/laboratory parameters

| | A2 | B2 | C2 |
|---|---|---|---|
| Cell viability + IL-15 + MCP-1 | Train AUC: 0.963 Test AUC: 0.719 (XGBCRUS) | Train AUC: 0.864 Test AUC: 0.736 (RFCRUS) | Train AUC: 0.695 Test AUC: 0.609 (LOGREGRUS) |
| Cell viability + IL-15 + MCP-1 + Baseline Hemoglobin | Train AUC: 0.967 Test AUC: 0.781 (XGBCRUS) | Train AUC: 0.903 Test AUC: 0.701 (RFCRUS) | Train AUC: 0.698 Test AUC: 0.616 (LOGREGRUS) |
| Cell viability + IL-15 + MCP-1 + Baseline Tumor Burden | Train AUC: 0.935 Test AUC: 0.779 (XGBCRUS) | Train AUC: 0.864 Test AUC: 0.710 (XGBCRUS) | Train AUC: 0.697 Test AUC: 0.598 (LOGREGRUS) |
| Cell viability + IL-15 + MCP-1 + Baseline LDH | Train AUC: 0.989 Test AUC: 0.701 (XGBCRUS) | Train AUC: 0.979 Test AUC: 0.748 (RFCRUS) | Train AUC: 0.786 Test AUC: 0.582 (RUSBoost) |
| Cell viability + IL-15 + MCP-1 + Baseline Creatinine | Train AUC: 0.835 Test AUC: 0.714 (RFCRUS) | Train AUC: 0.854 Test AUC: 0.723 (XGBCRUS) | Train AUC: 0.702 Test AUC: 0.629 (LOGREGRUS) |
| Cell viability + IL-15 + MCP-1 + Baseline Calcium | Train AUC: 0.931 Test AUC: 0.745 (RFCRUS) | Train AUC: 0.859 Test AUC: 0.743 (XGBCRUS) | Train AUC: 0.762 Test AUC: 0.604 (RUSBoost) |
| | A3 | B3 | C3 |
| Cell viability + IL-15 + MCP-1 | Train AUC: 0.803 Test AUC: 0.750 (RFCRUS) | Train AUC: 0.998 Test AUC: 0.757 (XGBCRUS) | Train AUC: 0.773 Test AUC: 0.766 (XGBCRUS) |
| Cell viability + IL-15 + MCP-1 + Baseline Hemoglobin | Train AUC: 0.867 Test AUC: 0.786 (BRFC) | Train AUC: 1.000 Test AUC: 0.710 (XGBCRUS) | Train AUC: 0.764 Test AUC: 0.708 (LOGREGRUS) |
| Cell viability + IL-15 + MCP-1 + Baseline Tumor Burden | Train AUC: 0.862 Test AUC: 0.740 (BRFC) | Train AUC: 0.858 Test AUC: 0.658 (XGBCRUS) | Train AUC: 0.768 Test AUC: 0.708 (LOGREGRUS) |
| Cell viability + IL-15 + MCP-1 + Baseline LDH | Train AUC: 0.876 Test AUC: 0.790 (BRFC) | Train AUC: 0.936 Test AUC: 0.715 (XGBCRUS) | Train AUC: 0.770 Test AUC: 0.708 (LOGREGRUS) |
| Cell viability + IL-15 + MCP-1 + Baseline Creatinine | Train AUC: 0.821 Test AUC: 0.757 (RFCRUS) | Train AUC: 0.941 Test AUC: 0.728 (XGBCRUS) | Train AUC: 0.751 Test AUC: 0.735 (XGBCRUS) |
| Cell viability + IL-15 + MCP-1 + Baseline Calcium | Train AUC: 0.835 Test AUC: 0.762 (RFCRUS) | Train AUC: 0.964 Test AUC: 0.805 (XGBCRUS) | Train AUC: 0.750 Test AUC: 0.744 (XGBCRUS) |
| | A4 | B4 | C4 |
| Cell viability + IL-15 + MCP-1 | Train AUC: 0.790 Test AUC: 0.808 (RFCRUS) | Train AUC: 0.859 Test AUC: 0.752 (RUSBoost) | Train AUC: 0.860 Test AUC: 0.620 (RFCRUS) |
| Cell viability + IL-15 + MCP-1 + Baseline Hemoglobin | Train AUC: 0.896 Test AUC: 0.764 (BRFC) | Train AUC: 0.953 Test AUC: 0.752 (XGBCRUS) | Train AUC: 0.814 Test AUC: 0.640 (XGBCRUS) |
| Cell viability + IL-15 + MCP-1 + Baseline Tumor Burden | Train AUC: 0.802 Test AUC: 0.779 (RFCRUS) | Train AUC: 0.914 Test AUC: 0.777 (BRFC) | Train AUC: 0.749 Test AUC: 0.582 (RUSBoost) |
| Cell viability + IL-15 + MCP-1 + Baseline LDH | Train AUC: 0.819 Test AUC: 0.800 (RFCRUS) | Train AUC: 0.913 Test AUC: 0.757 (BRFC) | Train AUC: 0.813 Test AUC: 0.645 (XGBCRUS) |
| Cell viability + IL-15 + MCP-1 + Baseline Creatinine | Train AUC: 0.909 Test AUC: 0.749 (RFCRUS) | Train AUC: 0.972 Test AUC: 0.755 (XGBCRUS) | Train AUC: 0.884 Test AUC: 0.606 (XGBCRUS) |
| Cell viability + IL-15 + MCP-1 + Baseline Calcium | Train AUC: 0.838 Test AUC: 0.751 (RFCRUS) | Train AUC: 0.986 Test AUC: 0.770 (RFCRUS) | Train AUC: 0.858 Test AUC: 0.614 (XGBCRUS) |

### Classification of Test Populations by Predictive Algorithms

Once the best covariates were identified, this example applied two approaches to classify test populations. The performance of the classification on test population was measured by confusion matrix.

Confusion Matrix: A confusion matrix for a classifier summarizes the number of correct and incorrect predictions by class in the form of a contingency table. A confusion matrix is useful to understand prediction accuracy of the classifier and the type of errors the classifier is more likely to make. Accuracy (accuracy represents the proportion of observations that are correctly classified to the true class, either positive or negative), Sensitivity (true positive rate) and Specificity (true negative rate) are calculated from the numbers in confusion matrix.

### Model Based Approach

This example applied the BPM on training data and obtained predicted probabilities, then made a ROC curve based on the predicted probabilities of subjects from training data and selected the optimal cut point as the cutoff value at which Youden’s index is largest (Youden’s index= sensitivity + specificity - 1). Subjects whose predicted probability above this cutoff value were classified as “outpatient”; others were classified as “inpatient”.

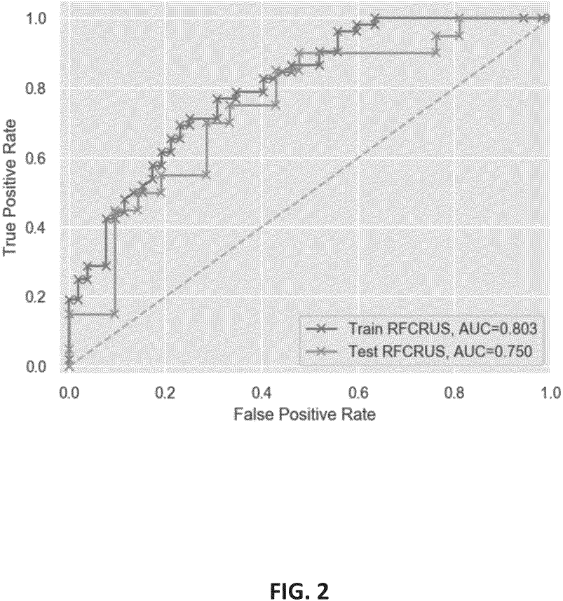

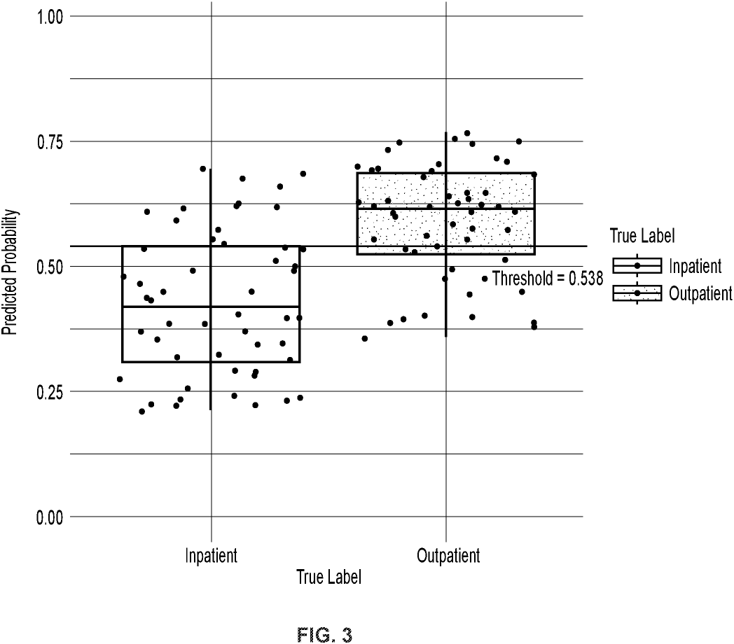

BPM for A3: For the minimalistic mechanistic model (use covariate of cell viability + Day 0 IL-15 + Day 0 MCP-1) on outpatient definition A3, this example chose Random Forest (RF) as the best performed algorithm. The ROC and box-plot of the BPM (RFCRUS; Optimal cut-off: 0.538) with Cell viability + IL-15 + MCP-1 on outpatient A3 is shown are FIGS. 2 and 3. The confusion matrix is shown in Table 7.

TABLE 7

Confusion Matrix on training data with cutoff=0.538

| | | Actual Class | |
|---|---|---|---|
| | | Inpatient | Outpatient |
| Predicted Class | Inpatient | 39 | 15 |
| | Outpatient | 13 | 37 |

Sensitivity: 0.7115, Specificity: 0.7500, Accuracy: 0.7308
Subjects with predicted probability>0.538 are classified as “outpatient”

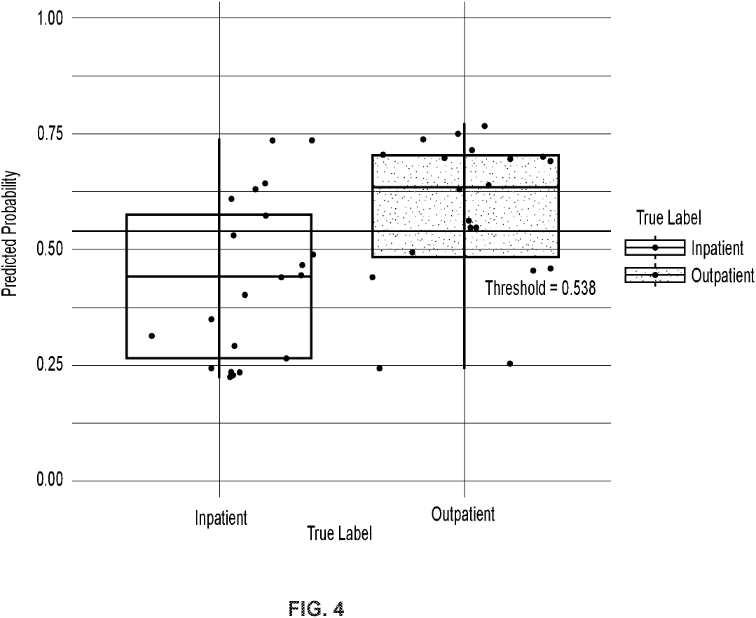

Box plot of predictions on testing data, BPM with Cell viability + IL-15 + MCP-1 on outpatient A3 is shown in FIG. 4. The confusion matrix is shown in Table 7.

TABLE 8

Confusion Matrix on testing data with cutoff=0.538

| | | Actual Class | |
|---|---|---|---|
| | | Inpatient | Outpatient |
| Predicted Class | Inpatient | 15 | 6 |
| | Outpatient | 6 | 14 |

Sensitivity: 0.7000, Specificity: 0.7143, Accuracy: 0.7073
Subjects with predicted probability>0.538 are classified as “outpatient”

### Tree Based Approach

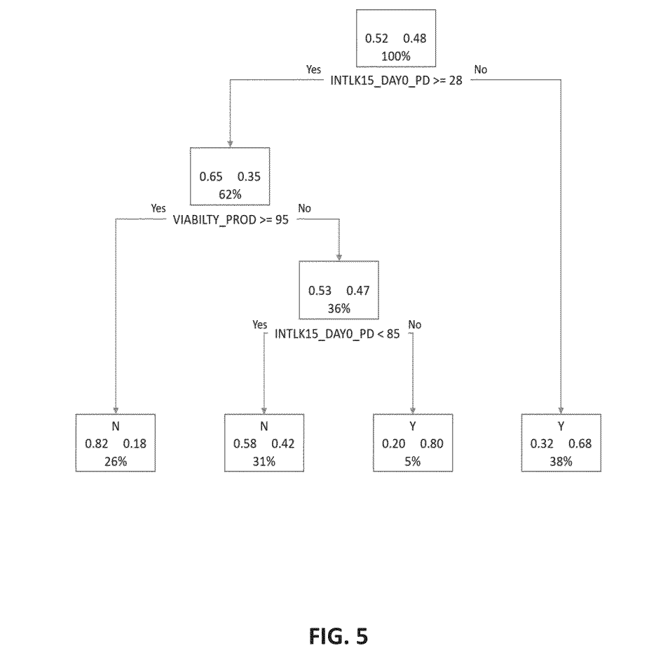

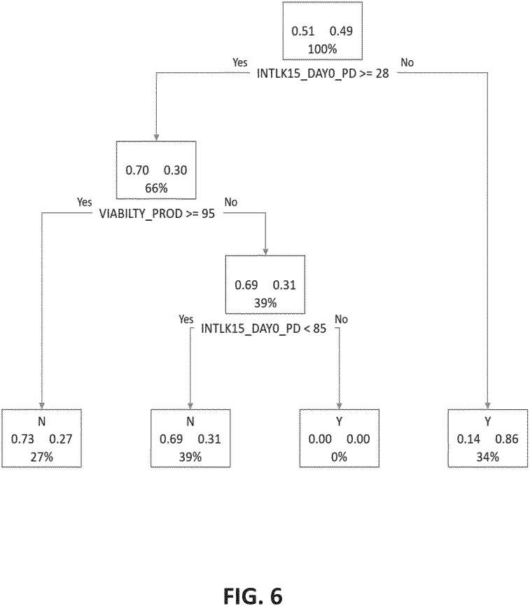

This example then built a decision tree by splitting selected best covariates in the training data, constituting the root node of the tree, into subsets which constitute the successor children. The splitting was based on a set of splitting rules based on classification features. The decision tree can be described as the combination of splitting on the selected best covariates to classify subjects to obtain high accuracy. The resulting decision trees are illustrated in FIG. 5 (training data) and FIG. 6 (testing data). The corresponding confusion matrices are shown in Tables 9 and 10.

TABLE 9

Confusion Matrix on training data on the decision tree in FIG. 5

| | | Actual Class | |
|---|---|---|---|
| | | Inpatient | Outpatient |
| Predicted Class | Inpatient | 42 | 19 |
| | Outpatient | 14 | 32 |

Sensitivity: 0.6275, Specificity: 0.7500, Accuracy: 0.6916

TABLE 10

Confusion Matrix on testing data on the decision tree in FIG. 6

| | | Actual Class | |
|---|---|---|---|
| | | Inpatient | Outpatient |
| Predicted Class | Inpatient | 19 | 8 |
| | Outpatient | 2 | 12 |

Sensitivity: 0.6000, Specificity: 0.9048, Accuracy: 0.7561

### Directionality

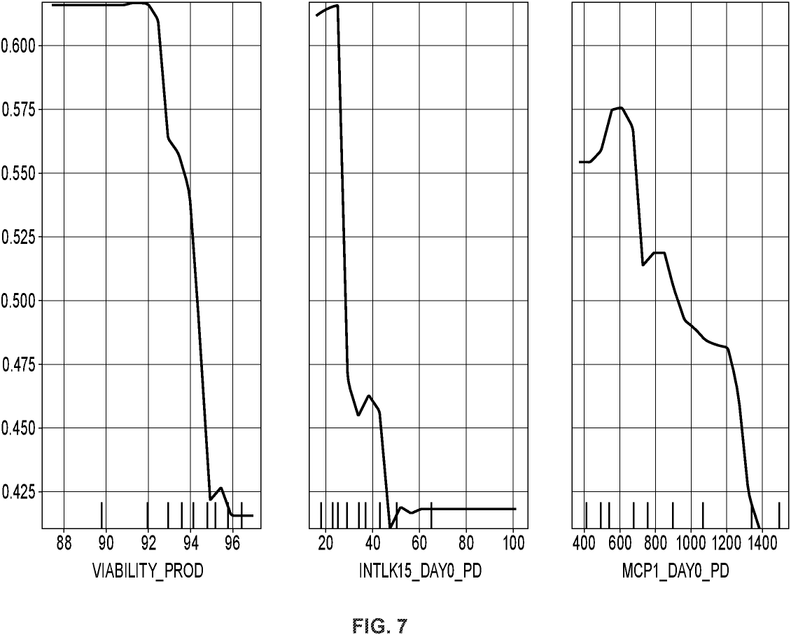

This example then used partial dependence plot to show whether the relationship between the onset toxicity and the covariate by leveraging out the effect of other covariates in the machine learning model. The plot is presented in FIG. 7. The plots suggest that a cutoff value for cell viability is at about 95%, a cutoff value for IL-15 is at about 28 pg/mL, and a cutoff value for CCL2 is at about 1300 pg/mL.

The directionality of covariates with onset of toxicity can also be presented by the estimated coefficients in a logistic regression of outpatient (Yes/No)~ Cell viability + IL-15 + MCP-1. The negative coefficients show that the three covariate mechanistic covariates all positively associated with early onset toxicities (Table 11).

TABLE 11

Estimated coefficients and associated p value from logistic regression of regression of outpatient (Yes/No)~ Cell viability + IL-15 + MCP-1

| | Estimated Coefficient | Associated p value |
|---|---|---|
| Cell Viability | -0.225 | 0.0068 |
| IL-15 at Day 0 | -0.00549 | 0.568 |
| MCP-1 at Day 0 | -0.00134 | 0.0307 |

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for identifying a patient as being likely or not likely to experience toxicity following a cell therapy, comprising:
measuring a level of at least one of IL-15 (Interleukin-15) and MCP-1 (monocyte chemoattractant protein-1) in a blood sample of the patient; and
identifying the patient as being likely to experience toxicity following the cell therapy when the level of IL-15 or MCP-1 is higher than a corresponding reference level, or identifying the patient as being not likely to experience toxicity following the cell therapy when the IL-15 or MCP-1 level is lower than a corresponding reference level,
wherein the cell therapy comprises administration of immune cells.

2. The method of claim 1, further comprising preventing or treating the toxicity in the patient, when the patient is identified as being likely to experience toxicity.

3. The method of claim 2, wherein the treatment or prevention comprises administration of an agent selected from the group consisting of anti-histamine, corticosteroid, antihypotensive agent, IL-6 inhibitor, GM-CSF inhibitor, and nonsteroidal anti-inflammatory drug.

4. The method of claim 3, wherein the treatment or prevention comprises administration of an agent selected from the group consisting of tocilizumab, dexamethasone, levetiracetam, lenzilumab, methylprednisolone, anakinra, siltuximab, ruxolitinib, cyclophosphamide, IVIG (intravenous immunoglobulin) and ATG (antithymocyte globulin).

5. The method of claim 1, wherein the immune cells comprise T cells engineered to express a chimeric antigen receptor (CAR).

6. The method of claim 5, wherein the CAR has binding specificity to a CD19 (cluster of differentiation 19) protein.

7. The method of claim 1, wherein the blood sample is a serum sample obtained from the patient prior to the cell therapy.

8. The method of claim 7, wherein the blood sample is obtained following a preconditioning treatment of the patient.

9. The method of claim 8, wherein the preconditioning treatment reduces lymphocytes in the patient.

10. The method of claim 1, wherein the toxicity is selected from the group consisting of cytokine release syndrome (CRS), neurologic events (NEs), and combinations thereof.

11. The method of claim 10, wherein the toxicity is early onset toxicity.

12. The method of claim 11, wherein the early onset toxicity occurs within four days following the cell therapy.

13. The method of claim 1, wherein the reference level for IL-15 or MCP-1 is determined from patients that experience the toxicity following the cell therapy and patients that do not experience the toxicity following the cell therapy.

14. The method of claim 1, further comprising measuring viability of cells used in the cell therapy, wherein the patient is identified as being likely to experience toxicity following the cell therapy when the IL-15 or MCP-1 level is higher than the corresponding reference level and the cell viability is greater than a reference cell viability, or wherein the patient is identified as being not likely to experience toxicity following the cell therapy when the IL-15 or MCP-1 level is lower than the corresponding reference level and the cell viability is lower than the reference cell viability.

15. The method of claim 1, further comprising obtaining one or more levels of baseline hemoglobin, baseline tumor burden, baseline LDH, baseline creatinine, and baseline calcium of the patient.

16. A method for preventing or treating toxicity in a patient undergoing a cell therapy, comprising:
identifying the patient as being likely or not likely to experience toxicity following a cell therapy, comprising:
measuring a level of at least one of IL-15 (Interleukin-15) and MCP-1 (monocyte chemoattractant protein-1) in a blood sample of the patient; and
identifying the patient as being likely to experience toxicity following the cell therapy when the level of IL-15 or MCP-1 is higher than a corresponding reference level, or identifying the patient as being not likely to experience toxicity following the cell therapy when the IL-15 or MCP-1 level is lower than a corresponding reference level, and
administering to the patient an agent that prevents or treats cytokine release syndrome (CRS) or neurologic events (NEs) if the patient has been identified as being likely to experience toxicity following the cell therapy.

17. The method of claim 16, wherein the agent is selected from the group consisting of anti-histamine, corticosteroid, antihypotensive agent, IL-6 inhibitor, GM-CSF inhibitor, and nonsteroidal anti-inflammatory drug.

18. The method of claim 16, wherein the agent is selected from the group consisting of tocilizumab, dexamethasone, levetiracetam, lenzilumab, methylprednisolone, anakinra, siltuximab, ruxolitinib, cyclophosphamide, IVIG (intravenous immunoglobulin) and ATG (antithymocyte globulin).

19. The method of claim 16, further comprising measuring viability of cells used in the cell therapy, wherein the patient is identified as being likely to experience toxicity following the cell therapy when the IL-15 or MCP-1 level is higher than the corresponding reference level and the cell viability is greater than a reference cell viability, or wherein the patient is identified as being not likely to experience toxicity following the cell therapy when the IL-15 or MCP-1 level is lower than the corresponding reference level and the cell viability is lower than the reference cell viability.

20. The method of claim 16, further comprising obtaining one or more levels of baseline hemoglobin, baseline tumor burden, baseline LDH, baseline creatinine, and baseline calcium of the patient.

21. A kit or package useful for identifying a patient as being likely to experience toxicity following a cell therapy, comprising polynucleotide primers or probes or antibodies for measuring the expression level of IL-15 and MCP-1 in a biological sample.
